(19) United States
(12) Patent Application Publication
(10) Pub. No.: US 2003/0195439 A1
(43) Pub. Date: Oct. 16, 2003

(54) Thermal pad and boot designed for applying hot or cold treatment

(76) Inventor: Ronald J. Caselnova, Staten Island, NY (US)

Correspondence Address: Rashida A. Karmali, PhD, 10th Floor, 99 Wall Street, New York, NY, 10005, US

(21) Appl. No.: 10/438,730

(22) Filed: May 16, 2003

Related U.S. Application Data
(63) Continuation-in-part of application No. 09/862,227, filed on May 22, 2001, now abandoned.
(60) Provisional application No. 60/207,753, filed on May 30, 2000.

(51) Int. Cl. A61H 7/00; A61F 7/00

(52) U.S. Cl. 601/15; 607/111; 607/114

(57) ABSTRACT

A therapeutic device and boot designed to provide hot or cold therapy to the foot of a patient. The boot has a sole, said sole has a top and bottom surfaces, said top surface has placed on it a plurality of thermal bumps. The thermal bumps have an elongated shape and are filled with a thermal substance, which is capable of maintaining a heated or cooled state. Once the thermal device or the boot are initially heated or cooled, the foot is placed therein. When the thermal bumps contact the foot, heating or cooling of the contacted area is accomplished, and will continue for a prolonged period of time.

### CROSS REFEFERENCE TO RELATED APPLICATION

This application is a continuation-in-part of application Ser. No. 09/862,227, filed May 22, 2001 for Therapeutic Boot Having Heat Retaining Protuberances (now abandoned), and provisional application Serial No. 60/207,753, filed May 30, 2001.

### FIELD OF THE INVENTION

This invention relates to a thermal boot device or pad made of a thermal material and having a top surface and a bottom surface, said top surface of the device having located on it, a plurality of raised, elongated, flexible bumps called thermal bumps, said bumps filled with a thermal gel. Specifically, the flexible thermal bumps filled with the thermal gel create a larger surface for cold or hot therapy, and enable better penetration of heat or cold to areas requiring treatment. For example, areas include, but are not limited to, joints, toes, ankle dorsal or plantar region, or any area of the body in need of heat or cold therapy. A variety of embodiments of the thermal pad device are provided in varying sizes, including boots, mitts, soles, entire boot for the human foot, or customized products for any part of the body in need of hot or cold therapy. The system may also be modified in shape, size, cost or material for use in pets and other animals.

### BACKGROUND TO THE INVENTION

The most prevalent conditions seen in podiatric practice are heel pain/plantar fasciitis, fungal nails, ingrown toenails and other nail problems, diabetic foot, ulcers, wound care, corns and calluses, bunions, athlete's foot, foot injury, arthritis of toes, flat fleet or fallen arches, bone spurs, hammertoes, warts or infection. A large number of these conditions require as part of the treatment regimen, application of hot/cold therapy. Currently, it is extremely difficult for an individual in need of hot or cold treatment to the foot, to find a thermal boot that would be suitable for effectively reducing swelling or pain, to reduce inflammation and/or induce healing. The devices currently sold on the market and described in prior art, such as the gel filled mitt for hand injuries or the bead filled boot for heat therapy have several disadvantages, for example, they are unsuitable for creating temperature changes rapidly and for prolonged periods, to provide relief or they are unsuitable to be applied to hard to reach affected areas. There is need for a thermal boot device that can effectively apply heat or cold treatments throughout the foot and/or the affected part of the foot. Hot and cold therapy devices have been subject of earlier patents, for example, U.S. Pat. No. 5,027,801, issued to Grim, discloses an orthopedic gel pad assembly including a layer of gel, a backing layer extending across the rear of the gel, and an orthopedic support means for holding the gel pad assembly against the injured body part of the user. This gel assembly is weight bearing and would not be suitable for use in the foot because in the foot, the gel pad would be bulky and difficult to keep in place, and the thermal pad would not reach difficult to reach structures in the foot.

U.S. Pat. No. 5,921,243 issued to Shakoor, describes a device for applying heat or cold therapy to a human foot having a plantar and a dorsum. The device is made of a flexible material that can wrap around the plantar and the dorsum of the foot, and the device has one pouch filled with a liquid located in the plantar region and a second liquid filled pouch located in the dorsum. The liquid in each of the pouches is used to apply heat or cold to the foot. Shakoor does not describe or suggest in any way that the liquid should be filled in a plurality of bumps, nipples or mini-pouches. The disadvantages of the Shakoor device are that liquid filled pouches in flexible material do not reach the toes, and the flat liquid filled pouches do not engage or come into contact with curves of the foot caused by bony protuberances of irregularly shaped bones in the foot such as navicular and metatarsal heads. This creates the problem of inadequate heat or cold exchange.

U.S. Pat. No. 3,595,244 issued to Kugler describes a foot-massaging sandal of flexible construction so that the toe portion may bend during walking, and the foot-contacting surface has irregularly disposed ridges which effect massaging action on the plantar surface of the foot. The disadvantage of this device is that it is constructed for massaging using ridges of various widths, heights and spacing but are solid and made of polyvinyl chloride or polyethylene. In other words, the ridges cannot be filled liquid and make this device unsuitable for hot or cold therapy.

Moreover, the device is designed so that the ridges cannot be placed over the first and fifth metatarsal heads to avoid painful massage of corns and calluses. Thus this device teaches away from the use of ridges or thermal bumps all over the foot region, or other areas in need for hot or cold therapy.

U.S. Pat. No. 5,607,749 issued to Strumor describes an acupressure massaging system including an array of spaced flexible and collapsible nipples extending vertically from the upper surface of a platform. The platform has air flow and air channels. The nipples have a collapsible accordion type structure, so that compression and movement of nipples creates a re-circulation or airflow effect through air flow channels and air flow holes to prevent suction and promote free movement of the nipples to exert an acupressure massaging counterforce on contacting surfaces. The disadvantage of Strumor's device is that it has nipples that are collapsible and because it is a weight-bearing device, it is unsuitable for use in applying hot/cold therapy because it cannot be filled with a thermal liquid.

There are other devices currently sold on the market, for example, the gel filled mitt used for hand injuries (having no thermal bumps but a smooth surface) and a boot for heat therapy, using beads to retain heat. However, these models also suffer from some of the disadvantages described above. Accordingly, the present invention overcomes the aforementioned disadvantages and is directed to a thermal boot device or pad, having a plurality of thermal bumps, each filled with a thermal gel, designed to enable improved penetration of heat or cold, to bring relief to areas requiring treatment. The invention provides specific products suitable for hot/cold therapy of foot conditions, as well as products that are developed for applying heat/cold therapy to the elbow, knee, ankle, neck, hand, wrist, shoulder, back, lumbar region, sinuses, temporo mandibular joint, head and pressure point areas where bed sores develop.

### SUMMARY OF THE INVENTION

The present invention is directed to a thermal boot device or pad having a top surface and a bottom surface, the top surface having configured on it, a plurality of raised, elongated, flexible thermal bumps, said thermal bumps being filled with a conventional source of thermal gel. The thermal bumps are uniquely configured of a suitable height and width to create a large surface area for heat or cold exchange and to enable better penetration of heat or cold to affected or injured areas requiring treatment. The thermal bumps are further distinctly configured to more efficiently cause a rapid and sustained temperature change to ensure that the optimum temperature desired is achieved faster and to create comfort for the individual. The present invention was conceived out of a need for it, by the inventor, because the inventor found the devices available on the market were not suitable in helping his patients fast enough. The inventor designed the present invention to solve the problem and assist the patients better.

The present invention also provides thermal boot including a thermal boot device or pad that can be incorporated in the dorsum portion of the thermal boot.

The present invention also provides a thermal boot having a thermal boot device pad incorporated in the plantar region.

The present invention also provides a thermal boot including the thermal boot device or pad incorporated allover the inside of the thermal boot. Optionally, the present invention provides a thermal boot device or pad custom made to size, which can be incorporated to selected area of the thermal boot, where corresponding foot parts are in need for hot or cold therapy.

Still further, the present invention provides a thermal boot device or pad that may be custom-designed to be used in any of the part of the body where hot or cold treatment is needed, for example, elbow, knee, ankle, neck, hand, wrist, shoulder, back, lumbar region, sinuses, temporo mandibular joint, head and pressure point areas where bed sores develop.

The invention also provides a therapeutic pad used for hot or cold therapy to the affected area of a patient. The thermal pad has a sole and an upper portion, whereupon a plurality of thermal bumps are located. The thermal bumps are made of or filled with a thermal substance which is capable of maintaining a heated or cooled environment around the affected area. When the thermal bumps contact the affected area, heating or cooling of the contacted area is effected, and will continue for a prolonged period of time.

It is an object of the present invention to produce a therapeutic boot which is capable of maintaining a cooled or heated state, so that is can be used to provide prolonged hot or cold therapy to a foot. Accordingly, the boot contains a thermal substance that has a high specific heat and thus a high thermal mass, such as a thermal gel, so that it is capable of maintaining a high or low temperature for a relatively long period of time.

It is another object of the invention to provide a therapeutic boot that facilitates healing by focusing its cooling or heating therapy directly at the joints. Accordingly, the thermal substance is contained within a plurality of thermal bumps or protuberances. The bumps direct pressure against the foot surfaces and thereby bring the thermal application directly to the affected joints. The thermal boot may be customized for a left or right foot, or may be in a generic shape suitable for either foot.

It is a further object of the invention to concentrate the heating or cooling effect on the area from the ankle to the toes. Accordingly, the bumps are concentrated in the area of the boot that directly corresponds to the area from the ankle to the toes when a foot is placed therein. Since circulation is commonly poorest to the feet, the boot provides complete heating/cooling therapy.

Accordingly, it is an object of the present invention to provide an inexpensive thermal pad device suitable for use in therapeutic hot or cold treatment, which can be incorporated within a wide variety and range of products and which is specially structured to apply hot or cold therapy in a wide variety of conditions besides foot therapy.

Still further, it is the object of the present invention to provide a thermal treatment pad made of a thermal material which includes an array of thermal bumps structured to provide penetration of heat or cold to a user upon contact so that the desired temperature is created to obtain relief from pain and recovery through healing in the affected area, for example, elbow, knee, ankle, neck, hand, wrist, shoulder, back, lumbar region, sinuses, temporo mandibular joint, head and pressure point areas where bed sores develop.

Still further, it is the object of the present invention to provide a thermal treatment pad which includes an array of thermal bumps structured to provide penetration of heat or cold to a user upon contact so that the desired temperature is created to obtain relief from pain and recovery through healing in the affected area for veterinary use.

To the accomplishment of the above and related objects the invention may be embodied in the form illustrated in the accompanying drawings. Attention is called to the fact, however, that the drawings are illustrative only. Variations are contemplated as being part of the invention, limited only by the scope of the claims.

### BRIEF DESCRIPTION OF THE FIGURES AND PREFERRED EMBODIMENTS

The advantages and features of the present invention will become readily apparent after reading the following detailed description and referencing the drawings. In the drawings, like elements are depicted by like reference numerals. In order to facilitate a fuller understanding of the present invention, reference is made of the drawings which should not be construed as limiting the present invention, but are intended to be exemplary only and , which are:

FIG. 1 is a diagrammatic perspective view of a boot according to the present invention per se.

FIG. 2 is a diagrammatic perspective view of just one embodiment of the boot sole.

FIG. 3 is a cross sectional view, taken generally in the direction of line 3-3 in FIG. 1.

FIG. 4 is a diagrammatic perspective view, illustrating the boot being prepared for hot therapy using a microwave oven.

FIG. 5 is a diagrammatic perspective view, illustrating a further embodiment of just the boot sole.

FIG. 6 is a diagrammatic perspective, illustrating yet another embodiment of the boot sole.

FIG. 7 is a diagrammatic perspective, illustrating two examples of the structure of the thermal bump.

### DETAILED DESCRIPTION OF THE INVENTION

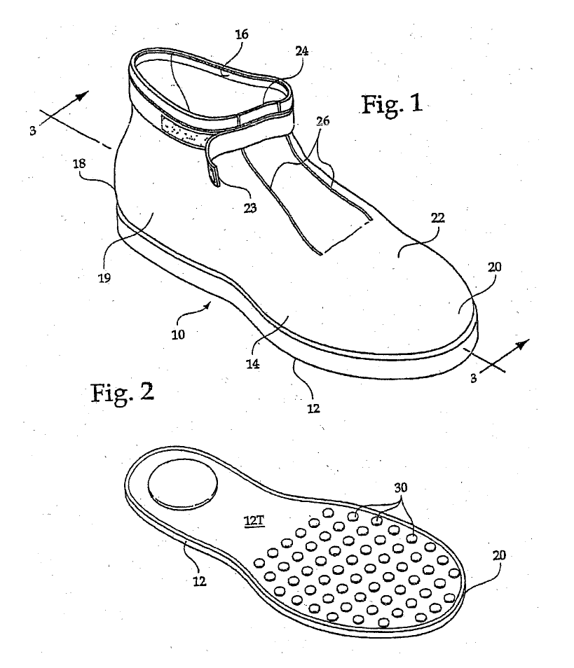

FIG. 1 illustrates a therapeutic boot 10, comprising a sole 12, and an upper portion 14. The upper portion 14 has an ankle opening 16. A tightening strap 22 is provided near the ankle opening 16 to facilitate securing onto the foot of a patient without trauma thereto. The upper portion 14, although generally continuous surface, can be described as having a rear portion 18, side portions 19, a forward portion 20, and a roof portion 22. The boot 10 is generally configured so as to accommodate a human foot with the toes thereof extending near the forward portion 20; the roof portion 22 covering the instep thereof, and the side portions 19 and rear portion 18 encasing the ankle thereof. The boot is configured so as to fully encase the foot to allow the patient limited motion while wearing the boot without compromising the therapeutic value thereof. However, the boot 10 is not intended to be weight-bearing, and thus is not necessarily configured to provide the same comfort and support features as ordinary footwear used for walking.

The roof portion 22 includes a tongue 24, which is formed by two longitudinal slits 26 which extends from the ankle opening 16 partially toward the forward portion 20. The longitudinal slits 26 extend substantially parallel to each other, and allow the boot 10 to adjust to accommodate different users. The tongue 24 also allows the boot 10 to be temporarily enlarged to allow insertion of an injured foot, which would otherwise have difficulty donning the boot 10. The tightening ankle strap 22 extends across the tongue 24 near the ankle opening 16 and allows the boot 10 to be secured onto the foot 10 after the tongue 24 has been suitably adjusted.

FIG. 2 illustrates the sole 12, which has a sole top surface 12T. In accordance with the present invention, a plurality of thermal bumps or protuberances 30 are located on the sole 12. The thermal bumps are placed on the top surface of the sole 12T in an area extending rearwardly from the forward portion 20. Further, the thermal bumps 30 are fabricated or filled with a thermal substance. The thermal substance has the properties of having a high specific heat, and thus of having a thermal mass. The thermal substance can be the commonly available “blue thermal gel”, commonly used in ice packs and the like, or it could be a similar substance. The thermal substance is therefore capable of being cooled or heated, and maintaining its cooled or heated state for a prolonged period of time. Thus, “thermal therapy” as used herein refers to the ability to maintain either a heated or a cooled state. Further, “heat retaining” as used herein refers to the ability to maintain either a heated or a cooled state. The thermal bumps are preferably approximately one half inch in diameter, and one half inch in height.

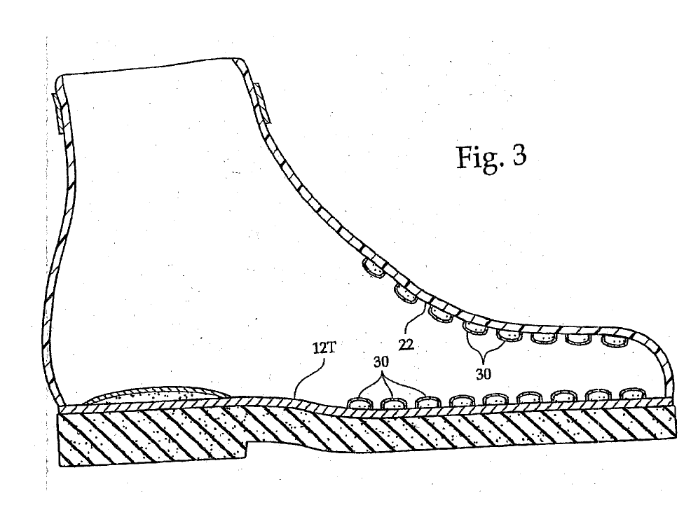

Most notable, referring to FIG. 3, thermal bumps 30 of the sole top surface 12T and roof portion 22 bring the protuberances 30 into contact with the toes and instep. An important difference in the present invention is in the design of the boot, for example, the top and bottom of the boot have raised thermal bumps filled with the thermal gel. These thermal bumps are elongated in structure and have a larger surface area than the smooth thermal pad described in prior art, and the present invention is therefore better suited to heat of cool the joints in contrast to putting one's foot or hand or any affected part into a smooth interior of a mitt or boot. Damage to the cartilage of a joint results in stiffness and pain due to inflammation. The thermal bumps of the present invention can better cool the area by coming into direct contact with the affected joints.

In a most preferred embodiment of the invention, the front and lower portions of the boot are covered with the thermal bumps that measure about half an inch in diameter and half an inch in height. In other embodiments, the size of the thermal bumps may be varied to carry out the job at hand. The thermal bumps are also pliable, flexible and durable so as to allow the patient to put on the boot comfortably.

In an alternate embodiment, the thermal boot may include a fastening means comprising one or more Velcro-like straps suitably attached at different locations, for example, at the ankle near the maleoli bone, at the top of the foot near the tarsal bone and metatarsal articulations or at the metatarsal head near the first phalanxes. The fastening means is generally sewn on from the lateral aspect to the undersole or plantar surface, thereby making the boot device unlike any in prior art, a non-weight bearing device.

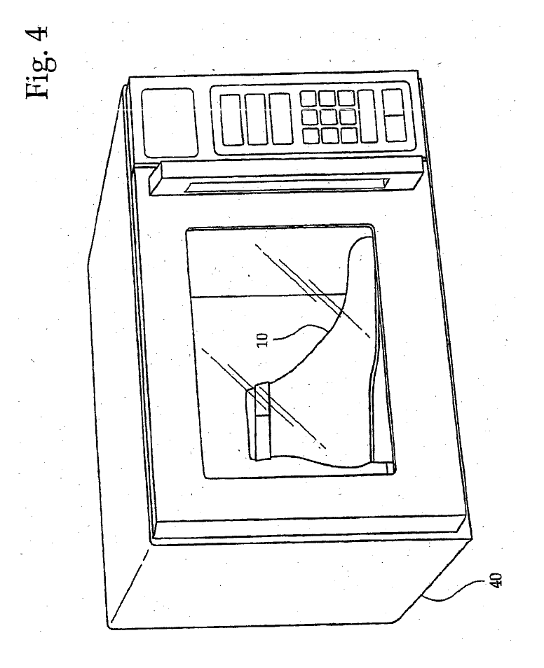

In use, the boot 10 is initially placed in a heating or cooling device 40, such as the microwave oven illustrated in FIG. 4 to effect heating of the boot, or a common refrigerator or freezer to effect cooling of the boot 10. The foot of the patient is then placed into the boot 10 through the ankle opening 16. When the foot is fully inserted into the boot 10, key portions of the foot are brought into contact with the thermal bumps. The thermal bumps are preferably pliable so that they bend as the patient puts the boot 10 on the foot. Additional thermal bumps 30 can be located to bring hot and cold therapy to the ankle, heel, or any other area of the foot deemed appropriate. Accordingly, the thermal bumps 30 are preferably disturbed throughout the interior of the boot 10, including on the underside of the tongue 24, to effect heating or cooling of the entire foot.

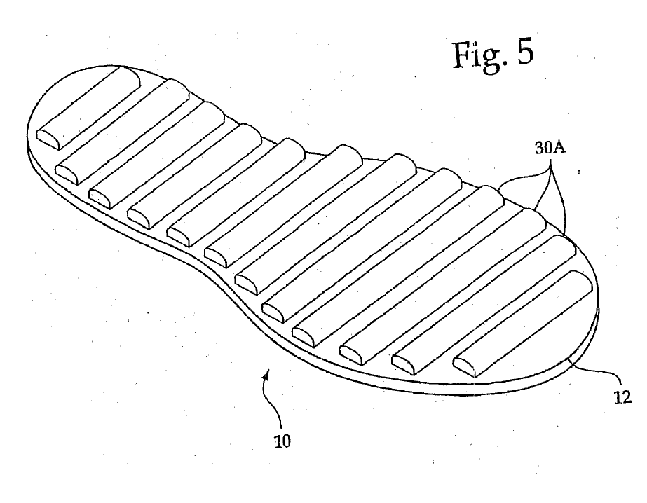

FIG. 5 illustrates a further embodiment of the sole 12 of the boot 10, wherein a series of transverse thermal bumps 30A are employed. In this embodiment, the protuberances are bar-like projections, which extend across the foot. Further experimentation will determine those configuration and patterns, which have the greatest therapeutic value. Such configurations can include mixing round and bar-like shaped thermal bumps within the same boot.

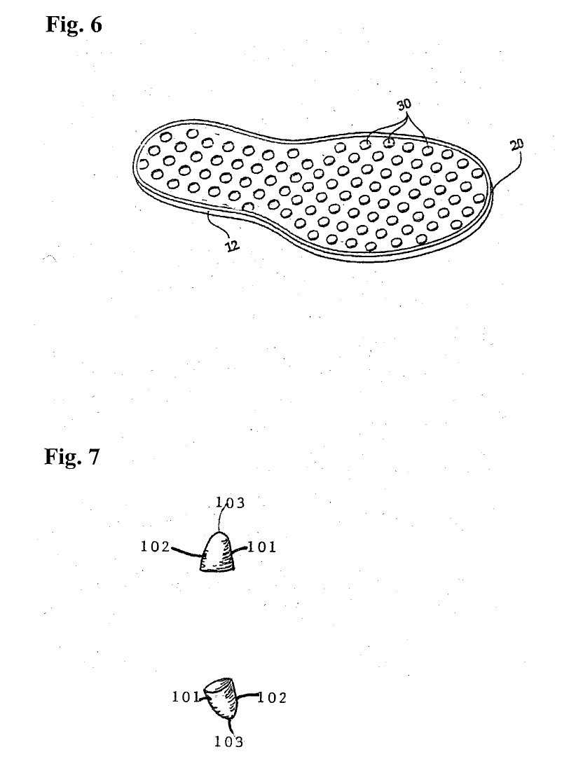

FIG. 6 illustrates another embodiment of the present invention wherein the sole 12 has on its top surface a plurality of thermal bumps placed throughout, extending from the region of the toes to the end of the heel portion.

FIG. 7 illustrated in detail the structure of the thermal bumps showing the unique elongated shape having two sides 101 and 103 and ending in a tapering end 103, said sides 101 and 102 creating a larger surface area than a flat pad, and said tapering end 103 allowing the thermal bump to reach difficult to reach areas in need of hot/cold therapy.

Once the foot is in place, the protuberances will provide the necessary hot or cold therapy to the foot for a prolonged period of time. The length of time for which the boot is effective is limited only by the properties and quantity of thermal substance used. The precise substance, which is best suited for meeting these goals, may be determined by empirical experimentation. Accordingly, the present invention is not limited to any specific thermal substance.

It should be noted that the principles of the boot according to the present invention apply equally well to other applications of the thermal device as incorporated into a variety of other products for hot/cold therapy, for example, in a mitt or glove, in wraps for the elbow, knee, ankle, neck, hand, wrist, shoulder, back, lumbar region, sinuses, temporo mandibular joint, head and pressure point areas where bed sores develop. That is, lining the interior surfaces of these various embodiments of the thermal device with the thermal device/thermal bumps which have heat retaining or losing characteristics would allow hot or cold therapy to be imparted to the affected area with similar benefits as can be achieved by the boot described in detail, which employs such thermal bumps.

In summary, herein is presented a therapeutic boot, which brings hot or cold therapy directly to the areas of the foot where it is most needed through the use of a plurality of protuberances made or filled with a thermal substance. The use of these protuberances increases the effectiveness of the hot or cold therapy, maintains contacts with the foot despite movement by the patient, and prolongs the period of effective therapy. The invention as described may be embodied in the form illustrated in the example provided in the drawing figures. Numerous variations are still possible while adhering to the spirit of the invention. Such variations are variations are still possible while adhering to the spirit of the invention. Such variations are contemplated as being part of the present inventive concept.

In a variety of additional embodiments the invention comprises of a thermal pad having a plurality of thermal bumps in selected areas or throughout the surface of the device, and such device being adapted in structure to be used as a therapy warp for sore muscles or joints such as tennis elbow and arthritis, sprained or pulled muscles. Another embodiment, a foot/ankle wrap may be used for sprained ankles from basketball or other running activities. A hot/cold pack available in assorted sizes may replace the electric healing pad, hot water bottle or a disposable cold pack. A cervical collar may provide therapeutic treatment to neck and upper cervical pain caused by injury, tension or muscle fatigue. A hand mitt provides excellent treatment for injured, over used or arthritic hands prior to and after rehabilitation exercises. A wrist wrap provides relief for carpal tunnel syndrome, sprains, strains or postoperative surgery and arthritis. A shoulder wrap uniquely designed to give treatment to muscles in the shoulder, upper arm, upper chest and upper back, is useful for baseball pitchers or any other athlete using the rotator cuff, pectorals or deltoids. The lumbar wrap relieves lower backaches and pains. The sinus mask uses cold to reduce swelling, numb the area and reduce pain, or it may be used hot to stimulate blood circulation and relief. In the embodiments described herein, the thermal device is cut to size and shape and covered with a breathable lycra-type shell that enables the dressing to conform to the curved area or bony prominences and is designed to relieve pressure and protect against friction.

The present invention is not to be limited in scope by the embodiment disclosed in the example which is intended as an illustration of one aspect of the invention and any methods which are functionally equivalent are within the scope of the invention. Indeed, various modifications of the invention in addition to those shown and described herein will become apparent to those skilled in the art from the foregoing description. Such modifications are intended to fall within the scope of the appended claims.

Those skilled in the art will recognize, or be able to ascertain using no more than routine experimentation, any equivalents to the specific embodiments of the invention described herein. Such equivalents are intended to be encompassed by the claims.

What is claimed is:

1. A thermal device for providing hot and cold therapy, said device comprising:
a thermal pad having a top surface and a bottom surface, said top surface having a forward and a rear portion, said top surface including a plurality of thermal bumps placed on it,
each of said thermal bumps is filled with a thermal gel capable of being cooled and heated to maintain a cooled state and a heated state, and
each of said thermal bumps having a structure capable of reaching areas difficult to reach to apply thermal therapy to an area in need of hot and cold therapy.

2. The thermal device according to claim 1, wherein the thermal pad is covered by a plurality of thermal bumps throughout the top surface of the thermal pads.

3. The thermal device according to claim 1, wherein said thermal bumps are selected from the group consisting of round, transverse shaped, bar-like projections, vertical and horizontal thermal bumps.

4. The thermal device according to claim 1, further comprising a support means suitable for applying hot/cold therapy to an area in need of hot/cold therapy.

5. The thermal device according to claim 1, wherein the thermal bumps are preferably half an inch in diameter and half an inch in height.

6. A thermal boot comprising a sole portion and a dorsal portion, said sole portion having a sole top surface, the upper portion having a forward and a rear portion, an ankle opening is located near the rear portion, the upper portion defining an interior which is configured so as to accommodate and fully enclose a human foot and ankle with the toes of said foot extending near the forward portion; and
a thermal pad located on the sole top surface, the thermal pad capable of selectively being cooled and heated and maintaining one of a cooled state and a heated state for a prolonged period of time so as to provide thermal therapy to the foot.

7. The thermal boot according to claim 6, further comprising a plurality of thermal bumps located on the sole, the thermal bumps having a thermal gel and suitably shaped to reach hard to reach areas in need of thermal therapy.

8. The thermal boot according to claim 6, wherein said dorsal portion further comprises a thermal pad.

9. A thermal device for providing hot and cold therapy, said device comprising:
a thermal pad having a top surface and a bottom surface, said top surface having a forward and a rear portion, said top surface including a plurality of thermal bumps placed on it,
each of said thermal bumps is filled with a thermal gel capable of being cooled and heated to maintain a cooled state and a heated state,
each of said thermal bumps having a structure capable of reaching areas difficult to reach to apply thermal therapy to an area in need of hot and cold therapy, and
a support means adapted to be attached to said thermal device to provide a wrapping means.

10. The thermal boot according to claim 9, wherein the wrapping means includes an elbow wrap, a knee wrap, an ankle wrap, a neck wrap, a hand wrap, a wrist wrap, a shoulder wrap, a back wrap, a lumbar region wrap, a sinus wrap, a temporo mandibular joint wrap, a head wrap or a wrap suitable for pressure point areas where bed sores develop.
